(19) United States
(12) Patent Application Publication
(10) Pub. No.: US 2013/0218010 A1
(43) Pub. Date: Aug. 22, 2013

(54) PORTABLE PET SCANNER FOR IMAGING THE HUMAN BRAIN

(71) Applicants: Weinberg Medical Physics LLC, (US); Irving N. Weinberg, (US); David Beylin, (US)

(72) Inventors: Irving N. WEINBERG, Bethesda, MD (US); David BEYLIN, Bethesda, MD (US)

(73) Assignees: Irving N. Weinberg, Bethesda, MD (US); WEINBERG MEDICAL PHYSICS LLC, Bethesda, MD (US); David Beylin, Bethesda, MD (US)

(21) Appl. No.: 13/772,579

(22) Filed: Feb. 21, 2013

Related U.S. Application Data
(60) Provisional application No. 61/601,302, filed on Feb. 21, 2012.

(51) Int. Cl. A61B 6/03; A61B 6/00

(52) U.S. Cl. CPC A61B 6/037; A61B 6/501; A61B 6/4405; A61B 6/481
USPC 600/431

(57) ABSTRACT

In accordance with at least one disclosed embodiment, an apparatus for use in imaging the human brain by describing the distribution of radiotracers in the human brain is provided. The apparatus may be compact and lightweight enough to be lifted by hand (less than 50 pounds) and affixed to a patient bed. In addition, the apparatus may collect images of both the cerebrum and cerebellum of the patient.

This application relies for priority on U.S. Provisional Patent Application Ser. No. 61/601,302, entitled “PORTABLE PET SCANNER FOR IMAGING THE HUMAN BRAIN,” filed on Feb. 21, 2012, the entirety of which being incorporated by reference herein.

### FIELD

Disclosed embodiments provide an apparatus for describing the distribution of radiotracers in the human brain and imaging the same.

### DESCRIPTION OF RELATED ART

Positron Emission Tomography (PET) is a nuclear medical imaging technique that produces a three-dimensional image or picture of functional processes in the body. A PET system detects pairs of gamma rays emitted indirectly by a positron-emitting radionuclide (tracer), which is introduced into the body as a component of a biologically active molecule. Typically, the gamma rays are detected by components configured in a round ring around the body of the patient (denoted in this document as “PET detector ring” or “PET ring”). Three-dimensional images of tracer concentration within the body are then constructed by computer analysis. In modern scanners, three dimensional imaging is often accomplished with the aid of a CT X-ray scan performed on the patient during the same session, while the patient lies on a single gantry that moves between the PET scan and CT scan detector rings.

Conventional PET scanners for human use that are compact and lightweight have been described for free-hand use, for example in the 2000 publication by I. N. Weinberg, V Zawarzin, R. Pani, G. DeVincentes, W. Worstell, entitled “Implementing reconstruction with hand-held gamma cameras”, as disclosed in the Conference Record of the 2000 IEEE Nuclear Science Symposium Vol. 3.

Many small PET scanners have been designed for animal use. An example is the RatCAP, a miniature PET scanner that can be mounted on a rodent's head, described in 2004 by P. Vaska, C. L. Woody, D. J. Schlyer, S. Shokouhi, S. P. Stoll, J. F. Pratte, P. O'Connor, S. S. Junnarkar, S. Rescia, B. Yu, M. Purschke, A. Kandasamy, A. Villanueva, A. Kriplani, V. Radeka, N. Volkow, R. Lecomte, and R. Fontaine, in a publication entitled “RatCAP: miniaturized head-mounted PET for conscious rodent brain imaging,” published in the IEEE Transactions on Nuclear Science, vol. 51, pages 2718-2722.

In a 2011 publication entitled “Development of a Brain PET System, PET-Hat: A Wearable PET System for Brain Research” in the IEEE Transactions on Nuclear Science, Vol. 58(3), pages 668-673, S. Yamamoto, M. Honda, T. Oohashi, K. Shimizu, and M. Senda disclose an apparatus in which a patient sits in a chair and the PET detector ring is lowered onto the patient's head.

### SUMMARY

Disclosed embodiments provide a compact and lightweight PET scanner, capable of being carried by hand, and can be rapidly affixed to, and removed from, a patient's bed or gantry or the like, upon which the patient lies. Typically the patient will lie horizontally.

In accordance with at least one disclosed embodiment, an apparatus for use in imaging the human brain by describing the distribution of radiotracers in the human brain is provided. The apparatus may be compact and lightweight enough to be lifted by hand (less than 50 pounds) and affixed to a patient bed. In addition, the apparatus may collect images of both the cerebrum and cerebellum of the patient.

In accordance with at least one disclosed embodiment, an additional aspect is the ability of the PET detector ring to simultaneously collect gamma rays arising from the cerebellum and the cerebrum of the patient.

Various other sources of utility will be made apparent from the discussion that follows and will be appreciated by those skilled in the art.

### BRIEF DESCRIPTION OF THE DRAWINGS

The invention will now be described in connection with the drawings appended hereto, in which:

FIG. 1 is a side-view of the PET ring as attached to the patient bed (or like horizontal surface).

FIG. 1a is a side view of the PET ring as attached to the patient bed (or like horizontal surface).

FIG. 2 is an end-on view of the PET ring as attached to the patient bed (or like horizontal surface) with human head positioned inside the PET ring.

FIG. 2a is an end-on view of the PET ring as attached to the patient bed (or like horizontal surface), where PET ring is connected with a computer and display unit either using cables or equipment for wireless communication.

FIGS. 3 and 4 are side-views of superior and inferior rings in an embodiment of the invention that minimally restricts patient vision.

### DETAILED DESCRIPTION

Disclosed embodiments will now be described in connection with one or more examples of embodiment implementations. It is intended for the embodiments to be representative of the invention and not limiting of the scope of the invention. The invention is intended to encompass equivalents and variations, as should be appreciated by those skilled in the art.

In accordance with at least one disclosed embodiment, the apparatus may be configured in a combined full and partial ring implementation. The full ring (denoted as the “superior ring”) completely encircles the superior portion of the brain (including the cerebrum) A less-than-full ring or partial ring (denoted as the “inferior ring”) outlines the inferior portion of the head (including the cerebellum). This less-than-full ring is denoted as the inferior ring. Tomographic PET image reconstructions may be performed using the superior ring.

It should be understood that the terms “PET detector ring” or “PET ring” encompasses the apparatus's superior and inferior rings or either the superior and inferior rings. Tomographic PET image reconstructions may be performed using detectors that are in the superior ring and/or in the inferior ring.

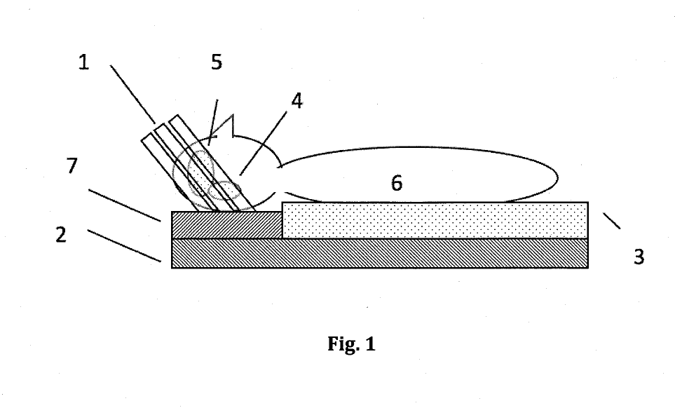

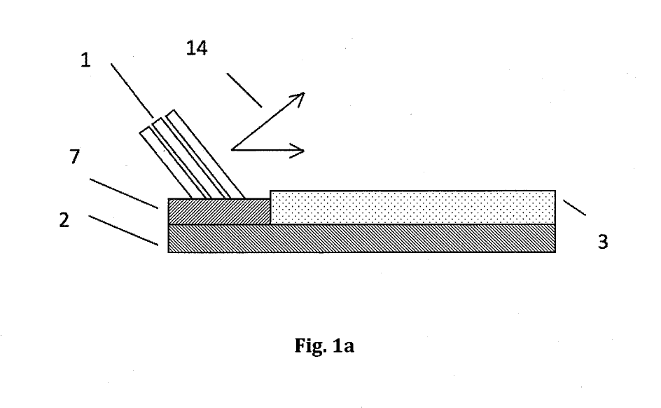

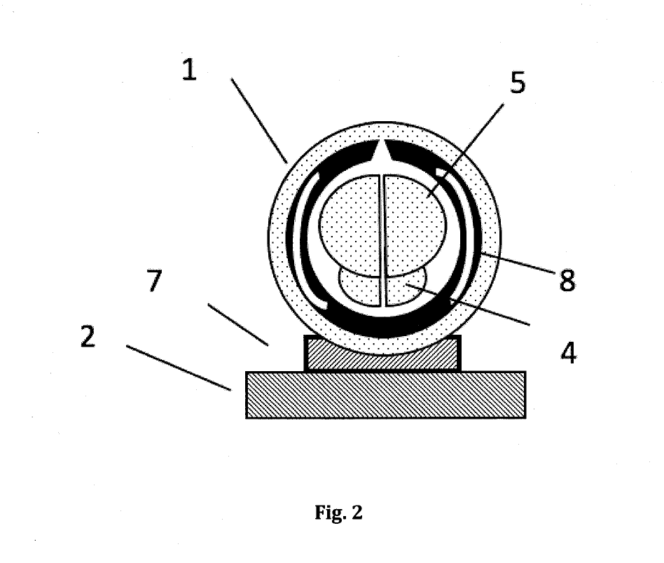

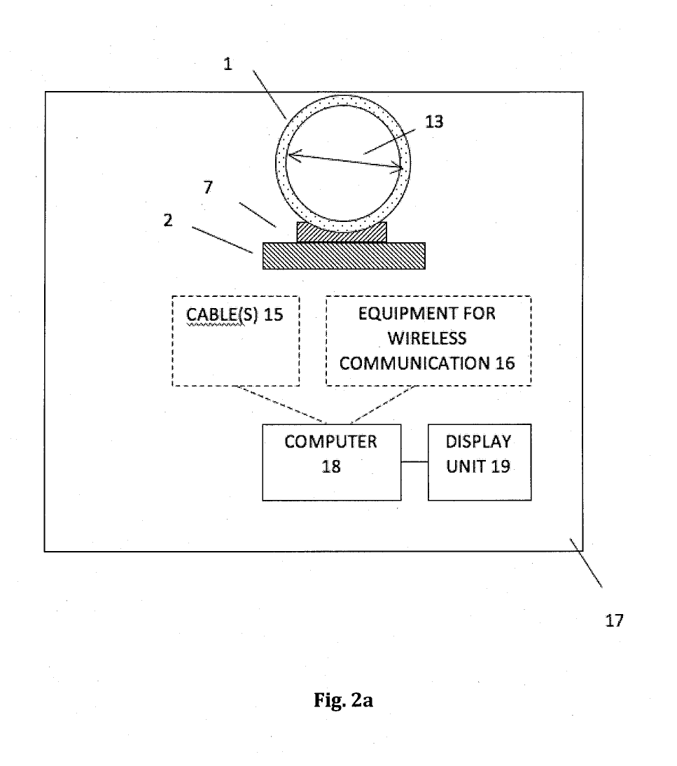

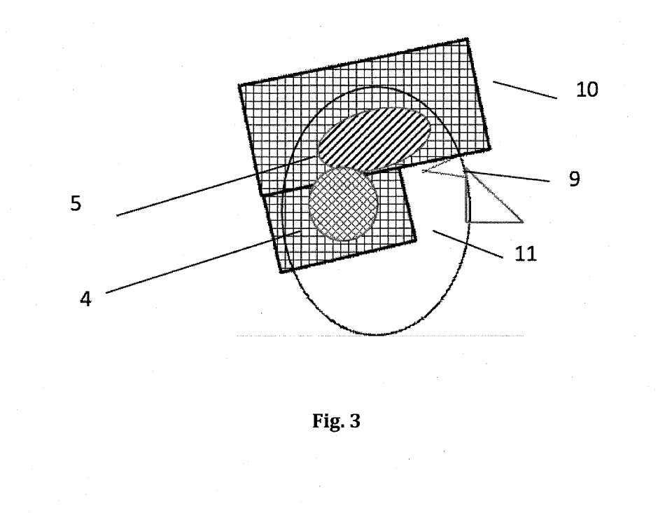

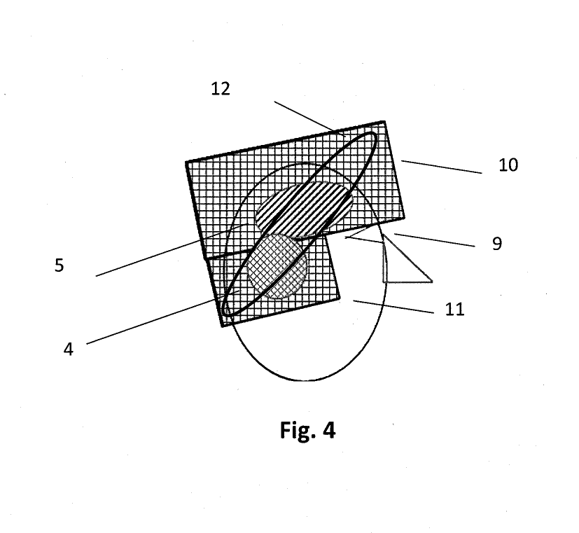

FIGS. 1, 1a, 2, and 2a are schematics of the disclosed embodiments (viewed from the side and top, respectively) showing the attachment of the PET detector ring 1 to the patient bed 2. Thus, FIG. 1 is a side-view of the PET detector ring 1 as attached to the patient bed 2 (or like horizontal surface) with patient 6 positioned on the bed. FIG. 1a is a side view of the PET ring as attached to the patient bed 2 (or like horizontal surface). FIG. 2 is an end-on view of the PET detector ring 1 as attached to the patient bed 2 (or like horizontal surface) with human head positioned inside the PET ring. FIG. 2a is an end-on view of the PET ring as attached to the patient bed 2 (or like horizontal surface). FIGS. 3 and 4 are side-views of superior and inferior rings in an embodiment of the invention that minimally restricts patient vision.

Although conventional PET scanners for human use that are compact and lightweight have been described for free-hand use, see, e.g., Weinberg et al., presently disclosed embodiments differ from conventional PET scanners in that the presently disclosed embodiments provide a PET scanner that may be affixed to a patient bed (or like horizontal surface) and is, therefore, not free-hand. Affixing the PET scanner to a patient bed prevents the PET scanner from falling and injuring the patient. Thus, the PET scanner operator can manually position the head of the supine patient in the field of view of a PET scanner prior to the PET scan, but does not need to manually support the patient or the PET scanner components during the scan. Thus, the PET scanner operator can engage in other activities, e.g., operating PET scanner computer keyboard. Positioning the patient on a bed rather than in a chair also ensures that if the patient loses consciousness or balance while positioned inside the PET scanner, the patient will not move or fall. This is particularly important because patients undergoing brain PET scan may have neurological symptoms and balance disorders.

Thus, the presently disclosed PET scanner can be affixed to, or removed from, a patient bed (or like horizontal surface), unlike previously disclosed PET scanners for imagining a brain of a human patient sitting in a chair (Yamamoto et al). Thus, the presently-described PET detector ring 1 may include a fastener 7 for attachment to a patient bed, table or other horizontal surface. The fastener 7 may include quick-release bindings for attachment of the PET detector ring to the patient bed, table or other horizontal surface, but are not shown in the figures.

In accordance with at least one disclosed embodiment, a cushion 3 may be included in the apparatus configuration to add to patient comfort.

The inner diameter 13 of the PET detector ring 1 is sufficient to accommodate the patient head, e.g., where an internal diameter of the PET detector ring 1 is between 15 cm and 35 cm.

Optionally, the PET detector ring can move either in the direction perpendicular to the plane of the PET detector ring 14, or in the direction parallel to the bed 2 (or like horizontal surface), thus potentially increasing the field of view of the PET scanner.

It should be understood that cables 15 and/or wireless transmissions 16 may be incorporated to connect the PET detector ring 1 to other components of a PET scanner 17, including a computer 18 and display equipment 19. Thus, software running on the computer 18 is configured to enable generation of three-dimensional images of tracer concentration within the patient's body, e.g., the brain.

As illustrated in FIGS. 3-4, the PET detector ring 1 encircles portions of both the cerebellum 4 and cerebrum 5 of the patient 6; thus, images of these areas may be useful in subsequent analysis of images for making a proper diagnosis of medical and/or health related conditions.

FIGS. 3 and 4 are schematic of an embodiment of the invention in which the cerebellum 4 is partially encircled by a less-than-full ring 11 and the cerebrum 5 is entirely encircled by a full ring 10. The full ring 10 is angled so that the patient's eyes 9 are not entirely blocked. FIG. 4 illustrates that data for a tomographic PET image reconstruction can be collected using detectors along oval 12, comprised of members of rings 10 and 11.

In accordance with the presently disclosed embodiments, the PET scanner includes a gasket that fits around the head of the patient. As shown in the Figures, the gasket 8 is also provided for immobilizing the patient in the PET detector ring. More specifically, the gasket 8 can be placed between the full ring (including the constituent superior and inferior rings) of the PET detector ring 1 and the patient's head, in order to immobilize the patient. The gasket 8 may optionally be inflatable.

The presently disclosed embodiments improve upon small PET scanners designed for animal use, see, e.g., Vaska et al., which are not designed to fit a patient's head, human or otherwise. Further, the presently disclosed embodiments differ from the conventional teachings of Yamamoto et al. in that the presently disclosed embodiments provide a PET scanner that is light-weight and can thus be carried by a person. Thus, in accordance with disclosed embodiments, a PET detector ring may be provided that is light-weight, e.g., weighing less than 50 pounds. Such a low-weight system allows the user to hand-carry the PET scanner between rooms in a healthcare or research facility or conveniently transport the scanner between healthcare or research facilities on a daily basis without employing a specially equipped van or truck.

Other aspects of the present invention should be apparent to those skilled in the art based on the discussion provided herein.

What is claimed is:

1. An apparatus for imaging positron-emitting radiotracers in human body, comprising:
a PET detector ring including a plurality of rings including, each including a plurality of PET detectors,
wherein data generated by the plurality of PET detectors can be used to perform tomographic PET image reconstruction.

2. The apparatus of claim 1, wherein the PET detector ring weighs less than 50 pounds.

3. The apparatus of claim 1, further comprising a computer running software configured to control the apparatus to generate three-dimensional images of tracer concentration within the patient's body.

4. The apparatus of claim 1, further comprising a fastener configured to attach the PET detector ring to a horizontal surface upon which a patent may lay, wherein the fastener is also configured to detach the PET detector ring from the horizontal surface.

5. The apparatus of claim 1, where the PET detector ring detects gamma-rays emanating from at least one portion of the cerebrum and at least one portion of the cerebellum.

6. The apparatus of claim 1, further comprising a gasket, wherein the PET detector ring is mechanically attached to the gasket and the gasket is configured to be in contact with a patient's head so as to immobilize the patient's head relative to the PET detector ring.

7. The apparatus of claim 1, where an internal diameter of the PET detector ring is between 15 cm and 35 cm.

8. The apparatus of claim 1, wherein the PET detector ring moves in a direction perpendicular to a plane of the PET detector ring during imaging by the apparatus.

9. The apparatus of claim 1, wherein the PET detector ring moves in a direction parallel to the horizontal surface during imaging by the apparatus.

10. The apparatus of claim 1, wherein the PET detector ring includes a plurality of detectors arrayed in a full and less than full ring, wherein a tomographic PET image reconstruction is generated by both the full and less than full rings.
